(19) United States
(12) Patent Application Publication
(10) Pub. No.: US 2002/0039771 A1
(43) Pub. Date: Apr. 4, 2002

(54) METHOD FOR PRODUCING COMPLEX MULTIENZYMATICAL, STORAGE RESISTANT REACTION MIXTURES AND USE THEREOF

(76) Inventors: LARS-ERIK PETERS, BERLIN (DE); PETER BENDZKO, BERLIN (DE)

Correspondence Address: NORRIS MCLAUGHLIIN & MARCUS P.A., 220 EAST 42ND STREET 30TH FLOOR, NEW YORK, NY, 10017, US

(21) Appl. No.: 09/230,070

(22) PCT Filed: Jul. 16, 1996
(86) PCT No.: PCT/DE96/01288

(51) Int. Cl. C12N 9/00; C12N 9/96

(52) U.S. Cl. 435/183; 435/188

(57) ABSTRACT

The invention describes a method and its use for producing complex multienzymatical, storage resistant reaction mixtures for synthesizing, modifying or analyzing polypeptides and optionally nucleic acids characterized in that native or artificial enzymatical, active protein mixtures with reaction buffers, cofactors and substrates are prepared so that they are ready for use and are storage resistant such that only user-specific key components (e.g. mRNS) are missing to start the desired enzymatical reaction(s). In the method a stabilizer is added to the reaction mixtures in the solution which, on the one hand, increases the reacting capacity of the multienzymatical systems and, on the other hand, protects the unstable reaction components from losing their biological activity or their biologically active structure while being made storage resistant and during storage. The reaction mixture is made storage resistant by being easily freeze dried under a vacuum and then durably stored at 4-10° C. (refrigerator temperature). Before use the user has to simply reconstitute the ready prepared reaction mixture by adding the original volume of H₂O and start the desired enzymatical reaction(s) by adding the user-specific component(s).

### DESCRIPTION

The invention describes a method for producing complex multienzymatic, storage stable reaction mixtures of native and, if necessary, artificial, enzymatically active protein mixtures ready for use which may be stored and transported at refrigerator temperature (0° C.-10° C.) without losing their activity and their use for synthesising, modifying or analysing polypeptides and nucleic acids.

The use of complex reaction systems consisting of cell extracts or enzyme mixtures for investigating the course of biochemical reactions is playing an ever more important part in modern biology and increasingly also in medical diagnostics.

Already for a long time problems relating to synthesising, folding, post-translational ripenening and inlanellular targeting of proteins have been investigated with the aid of cell-free extracts or lysates containing the complete ribosomal apparatus for the biosynthesis of proteins. In addition to applications in fundamental research multienzymatic reaction mixtures for a cell-free biosynthesis of proteins (in vitro translation) are gaining increasing importance for solving preparative-synthetic targets. Apart from that, in vitro translation has been used for synthesising protein fragments to map immunodominant epitopes, catalytic centres or substrate-binding sites. Since the so-called protein truncation assay (PTU) has been adopted for detecting relevant gene mutations an increasing demand for simply manipulatable, standardised reaction mixtures ready for use for the in vitro translation of synthetic mRNA produced through RT PCA has been stated in tumor diagnostics.

Also in the case of other analytical methods in molecular biology such as polymerase chain reactions (PCR), DNA sequencing and in vitro RNA synthesis (in vitro transcription) the trend to replace classical individual enzymatic assays by multienzymatic reaction systems is to be detected. Combining of a few enzymes and cofactors with specialised functions increases the processiveness and decreases the rate of mutating DNA and RNA polymerases in vitro. As a result, essentially longer DNA fragments (>10 kb) may be amplified than only by one enzyme. The accuracy of synthesis and product yield are higher, an invalid synthesis of non-specific by-products is better suppressed. Enzymes and protein factors combined in the PCR as a multienzymatic reaction system involve inorganic pyrophosphatase, DNA binding proteins, polymerase-specific antibodies and DNA polymerases with various exonuclease activities. Given the present state of the art the use of multienzymatic reaction mixtures is accompanied by a number of drawbacks as regards handling, reproducibility and storage stability retarding the use of multienzymatic reaction mixtures for the synthesis of proteins and DNA in applied research and diagnostics.

A decisive drawback of multienzymatic reaction systems as compared to single enzyme assays consists in the complicated handling and the limited or not existing storage stability. Complete reaction mixtures containing all enzymes, substrates and cofactors are not stable over longer periods in aqueous solutions neither at room temperature nor in a frozen state. This is a consequence of the fact that the conditions (pH, ionic strength, concentrations of the enzymes and the stabiliser, type of salts) which are optimal for carrying out the biochemical reaction differ from those required for preservation and reliably stabilization the enzyme components and cofactors (Franks, F. (1989) Process Biochem. 24 (1), R3-R7). In the case of multienzymatic reaction mixtures the problem is added that the individual components of cell extracts or enzymatic mixtures—depending on whether soluble enzymes, fibrillate structural proteins, membrane-associated enzyme complexes or nucleic proteins are concerned—make various demands on the stabilising medium and storage conditions. In most cases the different requirements are incompatible with each other. That is why commercial reaction systems in form of kits are providing the components individually are offered for in vitro translation, transcription, PCR or DNA sequencing. The following example of a reaction mixture for in vitro translation demonstrates how the various components of the kit have to be stored and handled under different conditions.

| Components of the reaction | | | |
|---|---|---|---|
| temperature | | | |
| mixture | | Factor of concentration | Storage |
| 1. | master mix (HEPES-KOH, ATP, | 12.5x | −20° C. |
| | GTP, DTT, tRNA, spermidine | | |
| | creatine phosphate) | | |
| 2. | mixture of amino acids (2.5 mM of | 50x | −20° C. |
| | each amino acid) | | |
| 3. | creatine kinase | 25x | 4° C. |
| 4. | RNase inhibitor | — | −20° C. |
| 5. | cell extract/lysate | 3x | −80° C. |
| | (cell-free extract, K acetate, Mg | | |
| | acetate, HEPES KOH, DTT) | | |
| 6. | translation buffer (K acetate/ | 25x | −20° C. |
| | Mg acetate) | | |

Each time before starting the experiment the user has to assemble the reaction mix from individual components. This stage of work is susceptible to errors and difficult to automate. With the number of parallel reactions the set-up time increases. Under the circumstances the set-up of the experiment takes often more time than its proper performance.

Because of the various temperatures required (4° C., −20° C. and −80° C.) the equipment required for storage and shipment of the starting components is extraordinary high. A kit for an in vitro translation has to be dispatched in dry ice to prevent the cell extract from thawing. The most sensitive and, at the same time, important component of in vitro translation assays is the cell extract with the macromolecular nucleoprotein complexes for ribosomal protein synthesis. The complex biochemical reaction of the RNA controlled synthesis of protein requires a co-operative interaction between a multitude of enzymes, enzyme complexes and structural proteins of a various structure and stability. In contrast to cells where macromolecular protein complexes of the ribosomal translation machinery are stabilised by the interaction with the filaments of the cytoskeleton cell-free extracts and lysates do not contain functional elements of the cytoskeleton. Free in solution macromolecular complexes dissociate easily, thus losing their activity.

Storage in a deep-frozen state at −80° C./−120° C. has been so far the only reliable method for preserving soluble biologically active cell extracts involving a number of problems and drawbacks. While being frozen cell extracts or lysates of wheat germs, reticulocytes or bacteria cells lose partly their original enzymatic activity caused by the formation of water crystals which damage many proteins irreversibly. By repeated freezing and thawing for subsequent experiments cell-free lysates of E. coli and reticulocytes lose nearly completely their translation activity. After each thaw and freeze cycle wheat germ extracts loose about 20-40% of their activity.

The instability of cell-free extracts to repeated cycles of freezing and thawing forces the user to consume them in an uneconomic way. To achieve reproducible experimental results each charge of the translationally active cell extract may be thawed only once. An imaginable alternative would be the storage of complete reaction mixtures in a deep-frozen state at −80° C. ready for use. Tests with translation assays based on wheat germs showed that freezing them once and storing them at −80° C. for one week leds to a loss of translational activity of 60% as compared with the freshly prepared control mixture.

Thus, concentrated cell-free protein extracts are comparatively storage stable at lowest temperatures, whereas reaction mixtures with respectively diluted cell extracts are not. In this connection, a decisive factor for stability is the high protein concentration in undiluted cell extracts.

Similar stability problems are arising for reaction mixtures for PCR, in vitro transcription and DNA sequencing. Freezing itself in concentrated aqueous solutions deactivates DNA and RNA polymerases completely. Thus, they are only storage stable at −20° C. in the presence of highly concentrated cryoprotectors. But glycerol (50%) which is usually used as cryoprotector and others (DMSO, polyethylene glycol) in high concentrations affect the PCR (primer annealing) and in vitro transcription (Crowe, L. M. and J. H. Crowe, Dev. Biol. Stand. 74, 285-294). In glycerin concentrations<50% the stability of enzymes in storage declines at −20° C.

Since the first publications relating to the preparation of cell-free extracts for the in vitro translation appeared in the mid-70-ies the method of preparation and stable storage has changed only marginally. An alternative to the described state of the art would be to stabilize them for storage by freeze drying. This method was successfully applied to stabilise liposomes and membrane fractions, individual enzyme preparations or partial reaction mixtures not containing an enzyme component, with special sugars or polyols in combination with bivalent metal ions or tensides preventing or limiting the denaturation of biomolecules due to loss of water.

The sugar trehalose in combination with the known cryoprotectors such as PEG or DMSO (19, 9, 10) proved to be especially suited for stabilising enzyme preparations for storage. This sugar is a natural metabolic product of many plants, insects and microorganisms being enriched inlanellularly under specific stress conditions (heat shock, dehydration, radioactive irradiation), ensures the survival of these organisms.

The technical problem on which the present invention is based is to provide a method of preparation avoiding the drawbacks of the state of the art as regards reactivity, storage stability, shipment and preparation of multienzymatic reaction mixtures ready for use for perfomring biochemical reactions. In particular, the method shall bring about products where the reaction products to be stored and transported need not be deep-frozen and the provision of reaction mixtures ready for use, i.e. containing all reaction components, will essentially reduce the expenditure of the user on experiments and the reproducibility.

The task is solved according to the invention by claims 1 and 9. The subclaims refer to special ways of execution.

The method according to the invention where native and artificial enzymatically active protein mixtures with the reaction components and a stabilizer which, on the one hand, increases the reactivity of the multienzymatic system and, on the other hand, protects the unstable reaction components against losing their biological activity or their biologically active structure while being stabilized for storage and stored, are combined in aqueous solution. Subsequently, they are converted to a storage stable state at 0°10° C. by freeze drying, with the quantity of the stabiliser resulting in an increase in the enzymatic activity of the reaction mixture equivalent to the quantity required for stabilising the complex multienzymatic reaction mixture for storage.

Cell extracts, cell lysates or fractions of them are used as native, enzymatically active protein mixtures.

A combination of individually purified single enzymes, cofactors and, if necessary, structural proteins, possibly of different origin, are used as artificial, enzymatically active protein mixtures.

Reaction components according to the invention are enzymatic and non-enzymatic cofactors, enzyme substrates, nucleotides and nucleosides or their oligomers, proteins, peptides, thiol compounds, RNA, DNA and, if necessary, derivates of each of the above substances individually or in combination.

Preferentially a sugar—preferably trehalose—providing in a ready-to-use reaction mixture optimum conditions for stabilising the unstable reaction components at a concentration of 8-12% (M/vol) in aqueous solution and, in addition, ensuring the maximum specific product yield of the multienzymatic reaction mixtures during synthesis is used as a stabiliser.

Multienzymatic reaction mixtures are lyophilized under vacuum using commercially available lyophilization equipment for 3-4 hours immediately being frozen in liquid nitrogen or a dry ice/alcohol bath, if necessary.

The method according to the invention concerns preferentially multienzymatic reaction mixtures for in vitro translation in wheat germ extracts and PCR. 50 μl reaction mixtures are prepared and freeze-dried according to the method in accordance to the invention (1^{st} example of execution). After various periods of storage the freeze dried reaction mixtures are reconstituted in water with the respective mRNA and, if necessary, a radioactively labelled amino acid being added. The reactivity of the reaction mixtures prepared and stored by this way has been detected by translation of various mRNA: dehydrofolate reductase (DHFR, 17.5 kD) and obelin (20 kD). The translation product was quantitatively detected by measuring the TCA-precipitable total radioactivity in 5 μl of the reaction mixture or by determining the enzymatic activity of DHFR in 10 μl of the reaction mix after 2 hours of incubation at 25° C. The translation product was qualitatively detected by means of gel electrophoresis in SDS PAG and subsequent autoradiography. The yield of translation in reconstituted freeze-dried reaction mixtures was always compared with the product yield in untreated reaction mixtures with and without trehalose to determine the efficiency of stabilisation for storage. The translation activity of the reconstituted reaction mixtures varied between 92-100% after 1-3 months at 4° C. as compared with the activity in the untreated control reaction mixture with 10% trehalose (FIG. 6).

The reactivity of reconstituted freeze-dried PCR reaction mixtures with an artificial enzyme mixture was checked in a RAPD PCR assay. The artificial enzyme mixture for the PCR consisted of Taq DNA polymerase, Deep-Vent® polymerase and inorganic Tth pyrophosphatase in a mixing ratio of 10:1:0.2 (units).

Thus, the method according to the invention allows to prepare native and artificial, enzymatically active protein mixtures with reaction buffers, cofactors and substrates ready for use by:

adding a stabiliser to the reaction mixtures in aqueous solution, which improves, on the one hand, the reactivity of the multienzymatic system and protects, on the other hand, the unstable reaction components against the loss of their activity or biologically active structure while being stabilised for storage;

by drying them under vacuum after freezing them in liquid nitrogen,

if necessary, by covering them by inert gas.

The storage stable reaction mixtures thus obtained are used, according to the invention, after reconstitution in water (“milli Q” quality) by adding user-specific key components according to the desired enzymatic reaction for the synthesis, modification or analysis of proteins, polypeptides or nucleic acids.

The reaction mixtures prepared according to the invention have the advantage that they may be stably stored and shipped at 0°-10° C. Thus, the high costs on equipment required for providing and maintaining an intense cooling plant necessary according to the usual present of the art is saved. A second advantage is that the reaction mixtures containing all necessary components are ready for use. Thus, the user may start the desired reaction only by adding one or, at most, two key components. This removes the drawbacks of the state of the art relating to:

simultaneously carrying out a big number of parallel experiments (mapping of epitopes);

the reproducibility of parallel and subsequent enzyme reactions,

the time necessary for the preparation of the experiment,

the susceptibility to errors during the preparation of complex reaction mixtures,

the automation of complex biochemical reactions on an analytical scale.

The present invention is based on the detection that trehalose increases the specific product yield in the in vitro translation with wheat germ extracts and PCR in addition to its known protective effect during dehydration. In untreated, i.e. freshly prepared translation reaction mixtures, the specific product yield (synthesized protein per mRNA used) goes up depending on the trehalose concentration as compared with the reactions not containing trehalose. A maximum product yield is obtained at 10% w/v (FIG. 1).

It was possible to validate the “enhancer” effect of trehalose in aqueous solution for a number of model proteins and various wheat germ extracts (FIGS. 2a and 2b). It is thus a universal phenomenon independent of a product.

The newly found effect is a unique property of trehalose. All other auxiliary agents investigated used—according to the state of the art—equally trehalose as effective cryoprotector or stabilizer for vacuum drying—inhibit the in vitro translation in the range of concentration required for storage (FIG. 3).

Apart from that, it turned out that the concentration of trehalose which is optimal for stabilisation for storage by freeze-drying corresponds to the optimum concentration in the translation reaction in aqueous solution (FIG. 4).

Also in PCR applications an “enhancer” effect of trehalose was detected. Similarly as in the case of in vitro translation the yield of the specific amplificates increases with the trehalose concentration whereas the amplification of non-specific DNA fragments is suppressed (FIGS. 5a/5b).

The trehalose effect is especially drastic in an aqueous solution when amplifying DNA fragments >10 kb in Tris HCL reaction buffers where a specific product will not be amplified without trehalose (FIG. 5c).

Accordingly, the present invention differs from the state of the art relating to the use of trehalose as a stabiliser in two essential points. First, the method according to the invention surprisingly allows to prepare complete, multienzymatic reaction mixtures ready for use with a few unstable protein components and not individual enzymes or partial reaction mixtures without storage stable enzymes and without losing activity. Secondly, by utilising the newly detected “enhancer” effect of trehalose the conditions in the method according to the invention may be optimised in a way as to enable trehalose as unique additive to ensure a sufficient (long-term) storage stability and, at the same time, to increase the activity of the reconstituted multienzymatic reaction mixtures. As a result of applying the method according to the invention the reactivity of reconstituted reaction mixtures is higher than in untreated, ‘fresh’ reaction mixtures not containing a stabiliser. Thirdly, in contrast with the known methods only lyophilized reaction mixtures are storage stable and only in the temperature range between 0° and 10° C. for at least 6 months without losing activity. A storage at temperatures>15° C. and <0° C. results in a complete loss of activity within 1 month.

Hereinafter the invention shall be explained by two examples:

### 1. EXAMPLE OF EXECUTION

Preparation of a storage stable translation-active reaction mixture based on wheat germ extract using the stabiliser trehalose

a) Preparation of a Reaction Mixture Ready for Use from Individual Components

The reaction mixture is set up ice (0° C.-4° C.) in a sterile 2.0 ml microcentrifuge tube (screw cap with rubber joint and flat bottom are important!) of the following individual components:

| order | components and composition | volume | |
|---|---|---|---|
| 1. | H₂O milli Q | 13 | μl |
| 2. | amino acid mixture (2.5 mM of each of the 20 amino | 2 | μl |
| | acids | | |
| 3. | master mix (312 mM HEPES KOH pH 7.6, 12.5 mM | 4 | μl |
| | ATP, 1.25 mM GTP, 100 mM creatinine phosphate, | | |
| | 625 μg/ml yeast tRNA, | | |
| | 3.125 mM spermidine, 25 mM DTT) | | |
| 4. | creatinine phosphokinase 1.4 mg/ml | 2 | μl |
| 5. | 1 M potassium acetate | 2 | μl |
| 6. | 25 mM Mg acetate | 1 | μl |
| 7. | 50% trehalose | 10 | μl |
| 8. | wheat germ extract (90-100 OD₂₆₀) | 16 | μl |

Mix carefully the components in the reaction vessel (no pretexting). The volume of the ready reaction mix is 50 μl before stabilisation for storage. The volumes of the individual components have to be proportionally changed for 25 or 100 μl mixes.

b) Stabilisation of Translation Mixtures for Storage by Freeze Drying (Lyophilisation)

Immediately after having been mixed the open 2.0 ml reaction mixtures are quickly frozen in liquid nitrogen (−120° C.) and incubated in liquid nitrogen for 5 minutes. Thereupon, the frozen reaction mixtures are as quickly as possible transferred into a lyophilisation chamber which is connected to a commercial vacuum oil pump. The vacuum pump shall run ahead by 30-60 minutes to produce a strong vacuum in the lyophilisation chamber immediately after the valve will be opened. In the laboratory of the inventors a lyophilisation plant of Heto-Lab was used. The reaction mixtures were lyophilised at room temperature (20-30° C.) for 3-4 hours. Upon completion of lyophilisation the vacuum chamber was carefully aerated with ambient air or, if necessary, with an inert gas. The air entrance valve is equipped with a sterile filter to avoid a microbial contamination of the lyophilised reaction mixtures. Thereupon, the reaction vessels are air-sealed under sterile conditions by screw caps and additionally sealed with parafilm. In the case of need, also glass ampoules may be used as vessels for freeze-drying which are then accordingly closed by melting.

In this state lyophilised reaction mixtures are stored protected from light in a refrigerator at 0°-4° C.

c) Reconstitution and in vitro Translation

The lyophilised reaction mixtures are placed on ice and dissolved in 48 μl of “milli Q” H₂O. The dried residues are immediately dissolved in water. Thereupon, 2 μl of mRNA solution (0.5-2 μg/μl) are added. By careful pipetting the reaction mix is mixed. The reconstituted reaction mix is incubated at 25° C. for 2-3 hours for an in vitro translation. In the case of the translation product being radioactively labelled with L (¹⁴C) leucine (or ³⁵S methionine) the reconstitution is carried in 44 μl of “milli Q” H₂O, 2 μl of mRNA and 4 μl of L-(¹⁴C) leucine solution. After approbiate incubation time of incubation the quantity of the translation product is determined in 5 or 10 μl of the reaction mix. The determination is performed either enzymatically (enzyme activity of DHFR) or by measuring of radioactive substances precipitable by acid (in cmp) according to standard procedures in a scintillation counter.

### 2. EXAMPLE OF EXECUTION

Preparation of a storage satble reaction mixture for long-range and RAPD-PCR on the basis of an enzyme mixture of Taq DNA polymerase, Pfu DNA polymerase and inorganic pyrophosphatase of Thermus thermophilus

a) Preparation of a Reaction Mixture of Individual Components Ready for Use

The reaction mixture is pipetted onto ice (0° C.-4° C.) in a sterile 0.5 ml PCR tube (suitable for a respective thermocycler) of the following individual components:

| order | components and composition | volume | |
|---|---|---|---|
| 1. | milli Q H2O | 30 | μl |
| 2. | 10x reaction buffer (500 mM tricine KOH, pH 9.2, | 5 | μl |
| | 160 mM (NH₄)₂SO₄, 0.1% Tween 20) | | |
| 3. | 50x dNTP mix (12.5 mM dATP, dGTP, dCTP, dTTP) | 1 | μl |
| 4. | 50 mM MgCl₂ | 1.5 | μl |
| 5. | Forward primer (10 pmol/μl) | 1 | μl |
| 6. | Reverse primer (10 pmol/μl) | 1 | μl |
| 7. | 50% trehalose | 10 | μl |
| 8. | gelatin (20 mg/ml) | 0.5 | μl |

Mix the components in the PCR tube and centrifuge them off. The volume of the ready reaction mix is 50 μl before stabilisation for storage. The key component for starting the PCR is the respective template DNA. The volumes have to be proportionally changed for 25 or 100 μl reaction mixes.

b) Stabilisation of PCR Mixtures for Storage by Freeze Drying (Lyophilisation)

In conformity with example 1b of execution.

c) Reconstitution and PCR

The lyophilised reaction mixtures are placed on ice and are dissolved in 48 μl of “milli Q” H₂O. The dried residues are immediately dissolved in water. Thereupon 2 μl of template DNA solution (5-50 ng/μl) are added. By careful pipetting the reaction mix is mixed. The reconstituted reaction mix is transferred directly from an ice bath onto a preheated thermocycler (94° C.). There follows a 2-4 minute denaturation stage, the one of the two PCR programs is started depending on the application:

| | | | | |
|---|---|---|---|---|
| RAPD PCR: | 94° C. | 20 sec. | long-range PCR: | 94° C. |

| | | | |
|---|---|---|---|
| | 10 sec. | | |
| 37° C. | 30 sec. | 65° C. | 20 sec. |
| 72° C. | 60 Sec. | 68° C. | 10 min. |

| | |
|---|---|
| 35 cycles | 25 cycles |

1. Upon termination of the program always 10 μl of the reaction mix are applied onto 0.8% TAE agrarose gel and analysed electrophoretically.

### LEGEND FOR THE FIGURES

FIG. 1. Yield of DHFR synthesis in untreated reaction mixtures at various trehalose concentrations at the time of maximum product accumulation (2 h), tested with various wheat germ extracts

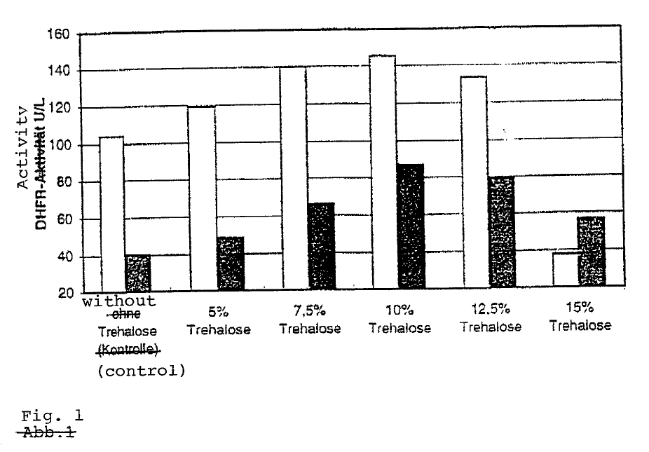

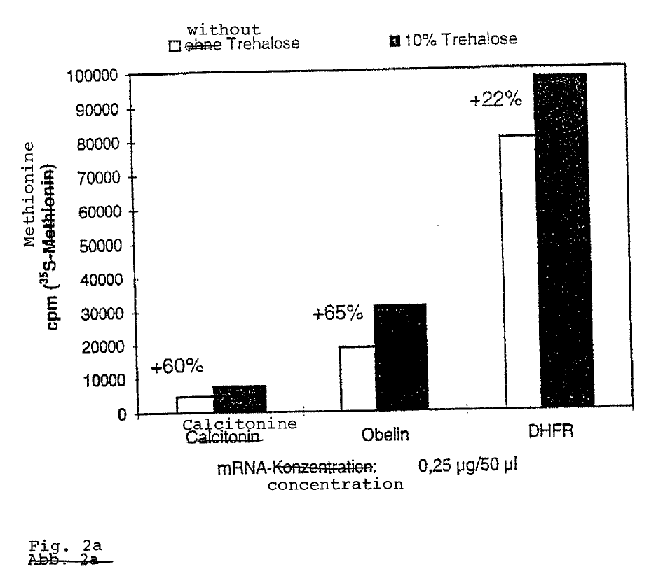

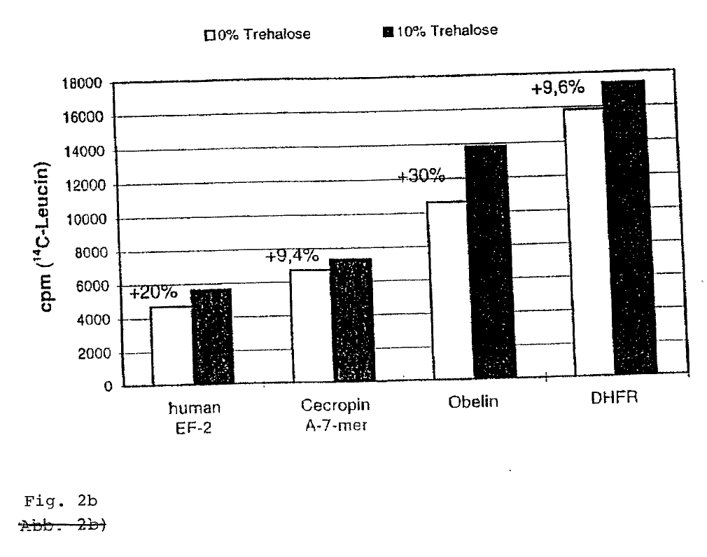

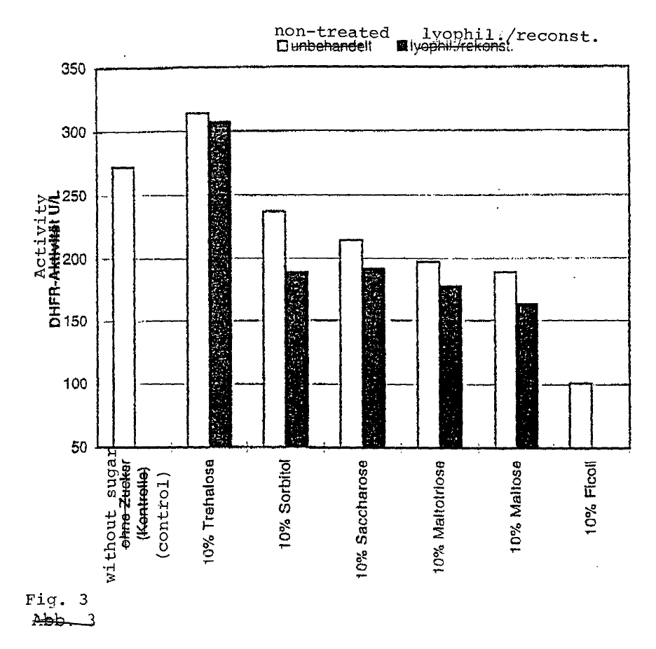

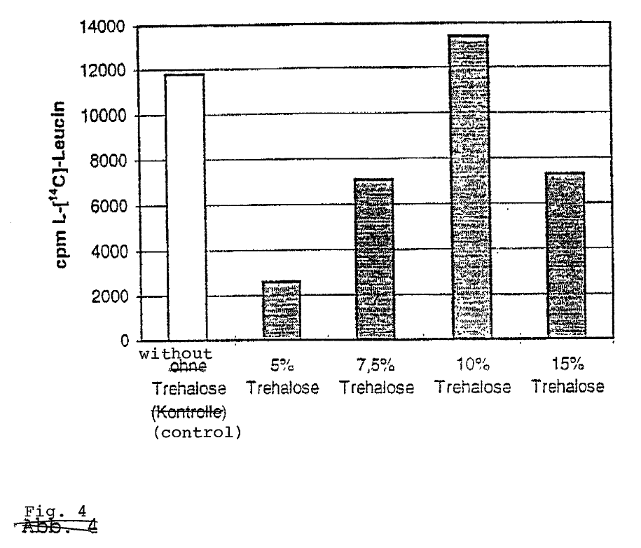

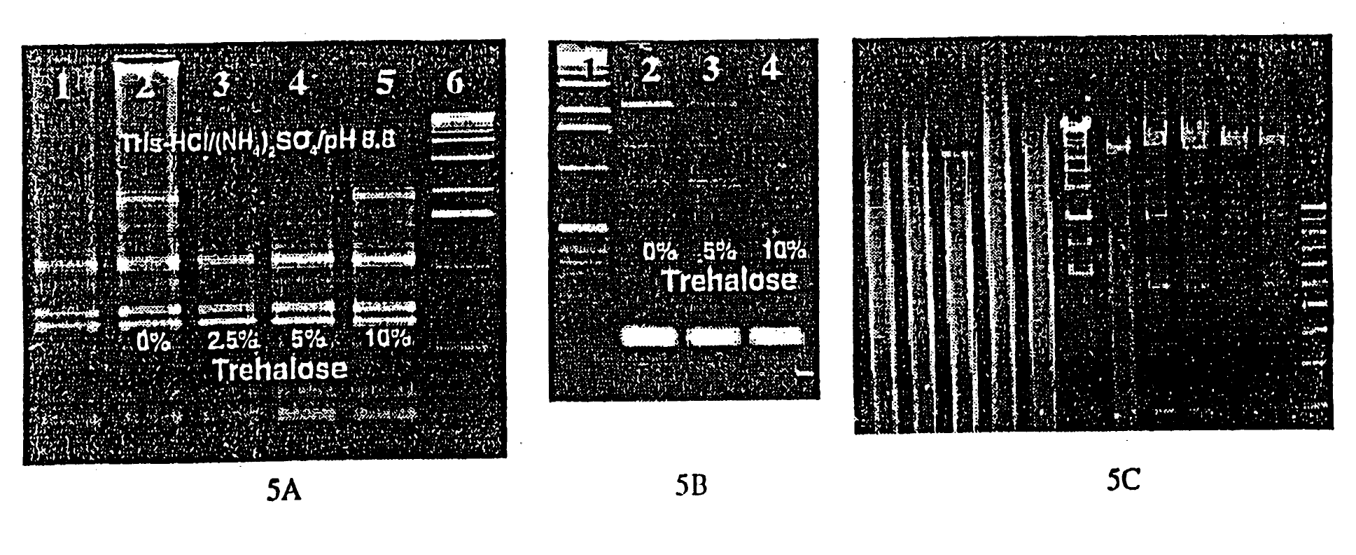

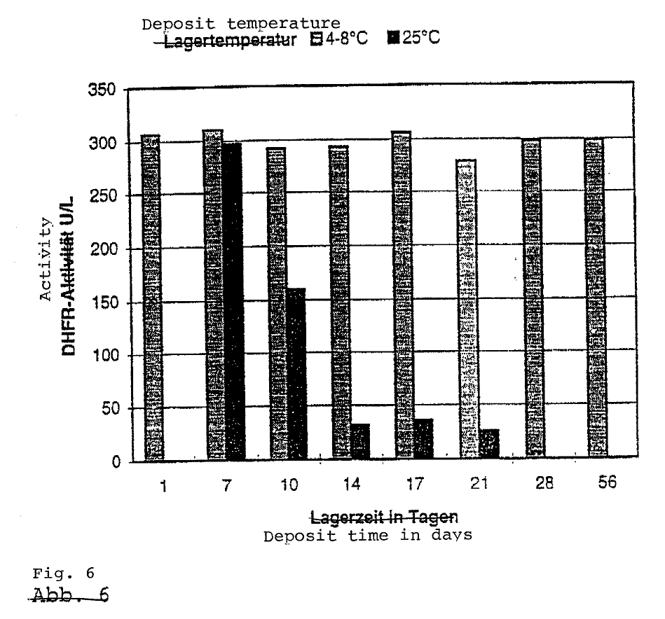

a) On the basis of highly active wheat germ extracts (lysate SL, white bars) and cell-free extract of less active wheat germs (lysate JB, black bars) 2 series of DHFR translation reactions (50 μl, 2 μg of DHFR mRNA) were prepared with increasing trehalose concentrations. After incubation of the in vitro translation for 2 hours at 25° C. the maximum product concentration was reached in the reaction mix (see translation kinetics in FIG. 6a). The product yield (DHFR activity) increases with the growing trehalose concentration reaching its maximum at 10% (M/vol). For the two wheat germ extracts the optimum trehalose concentration is about 10% in spite of various translation activities. In weaker active lysate shifting of the concentration dependence in favour of higher trehalose concentrations (12.5% and 15%) is noticed.

FIG. 2a “Enhancer” effect of trehalose in the in vitro translation of various mRNA in untreated reaction mixtures

Determination of the product yield (radioactive substances precipitable by acid) in 5 μl of translation reactions with 3 selected mRNA with and without addition of trehalose. Always 1 μl of [³⁵S]methionine (15 pmol=349.440 cpm) were used per reaction for the radioactive labeling of the translation product. 2 μl were taken from the reaction mixture after 2 hours for measuring the labelled translation products precipitable by acid. The following model proteins were investigated:

human calcitonine (120 bp, 0.25 μg),

obelin (700 bp, 0.25 μg),

E. coli DHFR (500 bp, 0.25 μg).

The translation reactions with labeling by [³⁵S]methionine were carried out at a subcritical RNA concentration. In the case of high RNA concentrations the synthesis reached its saturation point already after 15 minutes of incubation alue to the limited methionine concentration in the reaction.

FIG. 2b Demonstration of the positive trehalose effect in untreated radioactive translation assays with various model proteins being labelled by [¹⁴C]leucine

Comparison of product yield (radioactive substances precipitable by acid) in 50 μl of translation reactions with and without trehalose. Always 4 μl of [₁₄C]leucine (624 pmol=349.440 cpm) were used per reaction for the radioactive labeling of the translation product. 5 μl were taken from the reaction mixture after 3 hours to measur the labelled translation product precipitated by acid. The following model proteins were compared: human elongation factor 2 (hEF 2, 300 bp, 2.0 μg of RNA), an oligomer construct of the antibacterial peptide cecropin A (cecropin A-7-mer, 2.5 μg RNA), obelin (700 bp, 2.0 μg of RNA), E. coli DHFR (500 bp, 1.5 μg of RNA).

FIG. 3 Comparison of the effects of known stabilisers on the DHFR in vitro translation in untreated and reconstituted freeze-dried reaction mixtures

Two series of DHFR translation reactions were carried out under standard conditions (50 μl, 2 μg of DHFR mRNA, 2 h, 25° C.) always with a final concentration of selected sugars of 10% m/vol. The first series (white bars) of translation reactions consisted of untreated reaction mixtures mixed of individual components immediately before the in vitro synthesis was started. For the second series of experiments (black bars) complete reaction mixtures were prepared which, first freeze-dried and then reconstituted in 48 μl of bidest. water for starting the synthesis. A DHFR translation reaction was carried out in a standard reaction mixture without adding sugar for comparing the translation yield (DHFR activity). The DHFR activity was always determined from 10 μl of the reaction mix.

FIG. 4 Comparison of DHFR synthesis yield in reconstituted translation mixtures with various trehalose concentrations. Determination of optimum trehalose concentration for storage stabilisation.

After 3 hours of synthesis at 25° C. the maximum amount of the radioactively labelled translation product precipitated by acid was obtained (see translation kinetics in FIG. 3). As in the preceding experiment the highest product yield was reached at 10% (M/vol) of trehalose. A higher concentration (15%) inhibits the translation. The reduction of the translation yield at trehalose concentrations<10% as compared with the control reaction (untreated translation mixture not containing trehalose, not freeze-dried) is rather due to the insufficient stabilisation of the translationally active wheat germ lysate during freeze drying.

FIG. 5a Effects of trehalose on the performance of RAPD PCR assays

PAPD PCR with a 10-mer arbitrary primer and insect DNA in untreated reaction mixtures. The RAPD PCR was carried out under standard conditions with 250 ng of genomic DNA from Aeshna cynea.

By adding trehalose the non-specific background was reduced, an amplification of longer polymorphic DNA fragments (>2 kb) was brought about by false amplificates and intensified with the concentration increasing.

Lane 1: RAPD PCR in a standard PCR buffer [50 mM Tris HCl (pH 8.3 at 25° C.)—50 mM KCl—0.1% of triton 100].

Lane 2: RAPD PCR in a Tris PCR buffer II [50 mM Tris HCl (pH 8.8 at 25° C.)—16 mM (NH₄)₂SO₄-0.01% Tween 20].

Lanes 3-5: RAPD PCR in a Tris PCR buffer II, in combination with 2.5%, 5% and 10% of v/w trehalose.

FIG. 5b Effect of trehalose in untreated PCR reaction mixtures

Amplification of a 98 bp fragment from the cystic fibrosis gene under standard conditions. By adding trehalose to untreated reaction mixes the non-specific background (false amplificates up to 2 kb) is suppressed in dependance of the concentration.

Lane 1: DNA 1 kb ladder

Lane 2: PCR in a standard reaction mixture without trehalose

Lane 3: PCR in the presence of 5% trehalose

Lane 4: PCR in the presence of 10% trehalose.

FIG. 5c Effect of trehalose on long-range PCR in Tris HCl reaction buffers

Long-range PCR of a 20 kb λ DNA fragment in untreated reaction mixtures with various Tris HCl reaction buffers with and without trehalose, using an artificial enzyme mixture (Taq DNA polymerase, Pfu DNA polymerase, inorganic pyrophosphatase). Adding of 10% w/v of trehalose is a necessary and sufficient condition for the amplification of the specific DNA fragment under the described conditions.

Lane 1: LR PCR in a standard PCR buffer (Tris HCl/KCl/Triton X100, pH 8.3)

Lane 2: LR PCR in Tris HCl/(NH₄)₂SO₄/Tween 20 (pH 8.8)

Lane 3-5: LR PCR in commercial Tris HCl reaction buffers

Lane 6: High molecular weight DNA marker 9-48 kb (GIBCO BRL)

Lane 7-11: The same reaction mixes as in lanes 1-5, but with 10% trehalose

Lane 12: DNA 1 kb ladder (GIBCO BRL).

FIG. 6 Experiments relating to the long-term stability of freeze-dried translation assays

50 μl of translation reaction mixtures, completed by all components, except the DHFR mRNA, were freeze-dried under the known conditions and stored protected against light at two various temperatures. After various periods of time translation reactions were carried out under standard conditions (50 μl, 2 μl DHFR mRNA, 2 h, 25° C.) and the DHFR activity was determined. Always the same mRNA preparation was used for the translation reactions to determine exclusively the dependence of the translation activity of lyophilised reaction mixtures on the storage temperature and time.

Patent claims:

1. Method for producing complex multienzymatic storage stable reaction mixtures wherein native and, if necessary, artificial enzymatically active protein mixtures with reaction components and a stabiliser which increases, on the one hand, the reactivity of the multienzymatic system and, on the other hand, protects unstable reaction components from losing their biological activity or biologically active structure while being made storage stable and stored, are combined in aqueous solution and subsequently converted to a storage stable state at 0-10° C. by freeze-drying where the amount of the stabilizer providing an increase of the enzymatic activity of the reaction mixture is equivalent to the amount required for stabilization of the complex multienzymatic reaction mixture for storage.

2. Method according to claim 1 wherein cell extracts, cell lysates of fractions of them being used as native, enzymatically active protein mixtures.

3. Method according to claim 1 wherein a combination of purified single enzymes, cofactors and, if necessary, structural proteins which may be of various origin, are used as artificial enzymatically active protein mixtures.

4. Method according to claims 1 to 3 wherein enzymatic and non enzymatic cofactors, enzyme substrates, nucleotides and nucleosides or their oligomers, proteins, peptides, thiol compounds, RNA, DNA and, if necessary, derivates of each of the above substances are used individually or in combination as reaction components.

5. Method according to claims 1 to 4 wherein the stabiliser is a sugar with a concentration of 8-12% (M/vol) in aqueous solution.

6. Method according to claim 5 wherein the sugar is trehalose.

7. Method according to claims 1 to 6 wherein multienzymatic reaction mixtures are dried under vacuum with a commercial lyophylisation equipment at room temperature during 3-4 hours.

8. Method according to claim 7 wherein the reaction mixtures are frozen in liquid nitrogen or, if necessary, in a dry ice/alcohol bath immediately before being dried under vacuum.

9. Use of complex multienzymatic, storage stable reaction mixtures according to one of claims 1 to 8 for synthesising, modifying or analysing polypeptides or nucleic acids after reconstitution in water, with one or a few specific key components being added.

10. Use according to claim 9 wherein radioactively or not radioactively labelled amino acids or their respective aminoacyled tRNA molecules, radioactively or non-radioactively labelled nucleotides or, if necessary, their derivates or oligomers, natural or artificial messenger RNA, DNA of a various origin or combinations of the above substances are the key components.

11. Use according to claims 9 to 10 for cell-free ribosomal protein biosynthesis and, if necessary, for the post-translational modification of peptides, polypeptides and proteins.

12. Use according to claims 9 or 10 for replication, reverse or non-reverse transcription, if necessary, after enrichment or modification of nucleic acids in vitro.
